(19) United States
(12) Patent Application Publication
(10) Pub. No.: US 2024/0341731 A1
(43) Pub. Date: Oct. 17, 2024

(54) ULTRASOUND DIAGNOSTIC APPARATUS, NON-TRANSITORY COMPUTER-READABLE RECORDING MEDIUM STORING ULTRASOUND DIAGNOSTIC PROGRAM, AND CONTROL METHOD OF ULTRASOUND DIAGNOSTIC APPARATUS

(71) Applicant: KONICA MINOLTA, INC., Tokyo (JP)

(72) Inventor: Makiko URABE, Kanagawa (JP)

(73) Assignee: KONICA MINOLTA, INC., Tokyo (JP)

(21) Appl. No.: 18/631,503

(22) Filed: Apr. 10, 2024

(30) Foreign Application Priority Data
Apr. 17, 2023 (JP) 2023-067345

(51) Int. Cl. A61B 8/00; A61B 8/08

(52) U.S. Cl. CPC A61B 8/54; A61B 8/0883; A61B 8/469; A61B 8/5207

(57) ABSTRACT

An ultrasound diagnostic apparatus includes: an ultrasound probe configured to transmit and receive ultrasound to and from a subject; and one or more hardware processors; in which the hardware processor generates image data based on a reception signal received by the ultrasound probe, performs first measurement of performing real-time automatic measurement on the image data, and enables second measurement different from the real-time automatic measurement at a timing when the real-time automatic measurement performed in the first measurement has not been completed.

### CROSS REFERENCE TO RELATED APPLICATIONS

The entire disclosure of Japanese Patent Application No. 2023-067345 filed on Apr. 17, 2023 is incorporated herein by reference in its entirety.

### BACKGROUND

### Technological Field

The present invention relates to an ultrasound diagnostic apparatus, a non-transitory computer-readable recording medium storing an ultrasound diagnostic program, and a control method of an ultrasound diagnostic apparatus.

### Description of Related Art

An ultrasound diagnostic apparatus is known that transmits and receives ultrasound to and from a subject such as a living body using an ultrasound probe and generates an ultrasound image for diagnosis based on a signal obtained from an ultrasound echo due to reflection of the ultrasound.

In an ultrasound diagnostic apparatus, a generated ultrasound image can be displayed in real time, but when, for example, automatically measuring the amount of blood flow or the like, the ultrasound image being displayed in real time is frozen to be a static image for automatic measurement (e.g., Japanese Unexamined Patent Publication No. 2022-66592).

In the field of ultrasound diagnostic apparatuses, there has also been developed a real-time automatic measurement technique for automatically measuring, for example, a vascular radius in real time without freezing an ultrasound image being displayed in real time (e.g., Japanese Unexamined Patent Publication No. 2006-187484).

In a case where a moving organ such as a heart is automatically measured by freezing an ultrasound image displayed in real time, it is necessary to automatically measure the organ over a plurality of still images of the ultrasound image, and it takes time and effort to confirm a dynamic change.

On the other hand, in the case of the above-described real-time automatic measurement, automatic measurement may not be successful due to an unclear ultrasound image or the like, and in that case, there has been a problem that automatic measurement processing (region detection, boundary detection, or the like) is continued indefinitely.

In the case where automatic measurement processing is continued indefinitely, it is necessary to end the real-time automatic measurement by the decision of the user. For example, generally, after the start of the real-time automatic measurement, the user can end the real-time automatic measurement by pressing an “EXIT” button or the like, and then the user selects the manual measurement.

As described above, when the user presses the “EXIT” button or the like, the real-time automatic measurement can be ended, but when the user wants to continue the measurement itself, it is necessary to newly perform an operation of starting the manual measurement from the beginning, which takes extra time and effort for the measurement.

### SUMMARY

An object of the present invention is to provide an ultrasound diagnostic apparatus capable of stopping automatic measurement processing in a case where automatic measurement is not successful in real-time automatic measurement, a non-transitory computer-readable recording medium storing an ultrasound diagnostic program, and a control method of an ultrasound diagnostic apparatus.

In order to achieve at least one of the above-described objects, an ultrasound diagnostic apparatus includes an ultrasound probe configured to transmit and receive ultrasound to and from a subject; and one or more hardware processors; wherein the hardware processor generates image data based on a reception signal received by the ultrasound probe, performs first measurement of performing real-time automatic measurement on the image data, and enables second measurement different from the real-time automatic measurement at a timing when the real-time automatic measurement performed in the first measurement has not been completed.

In order to achieve at least one of the above-described objects, a non-transitory computer-readable recording medium storing an ultrasound diagnostic program for causing a computer to execute: transmission/reception processing of transmitting and receiving ultrasound to and from a subject with an ultrasound probe; generation processing of generating image data based on a reception signal received by the ultrasound probe; first measurement processing of performing first measurement of performing real-time automatic measurement on the image data; and second measurement processing of enabling second measurement different from the real-time automatic measurement at a timing when the real-time automatic measurement performed in the first measurement is not completed.

In order to achieve at least one of the above-described objects, a control method of an ultrasound diagnostic apparatus configured to transmit and receive ultrasound to and from a subject with an ultrasound probe and generate image data based on a reception signal received by the ultrasound probe, the control method including: performing first measurement of performing real-time automatic measurement on the image data; and enabling second measurement different from the real-time automatic measurement at a timing when the real-time automatic measurement performed in the first measurement is not completed.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is an external view illustrating an example of an ultrasound diagnostic apparatus according to an embodiment of the present invention;

FIG. 2 is a block diagram illustrating an example of the main part of the ultrasound diagnostic apparatus illustrated in FIG. 1;

FIG. 3 is a flowchart illustrating an example of a control method of the ultrasound diagnostic apparatus according to the embodiment of the present invention;

FIG. 4 is a flowchart illustrating another example (modification 1) of the control method of the ultrasound diagnostic apparatus according to the embodiment of the present invention;

FIG. 5 is a diagram illustrating an interruption operation in the flowchart shown in FIG. 4; and

FIG. 6 is a flowchart for explaining another example (modification 2) of the control method of the ultrasound diagnostic apparatus according to the embodiment of the present invention.

### DETAILED DESCRIPTION OF EMBODIMENTS

Hereinafter, one or more embodiments of the present invention will be described with reference to the drawings. However, the scope of the invention is not limited to the disclosed embodiments.

Hereinafter, embodiments of the present invention will be described in detail with reference to the drawings.

### Ultrasound Diagnostic Apparatus

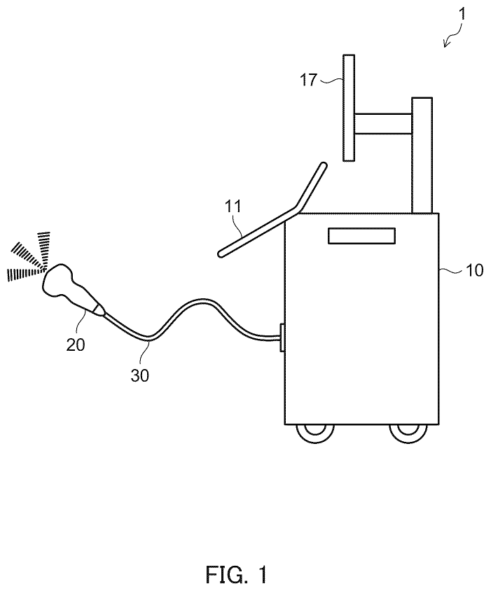

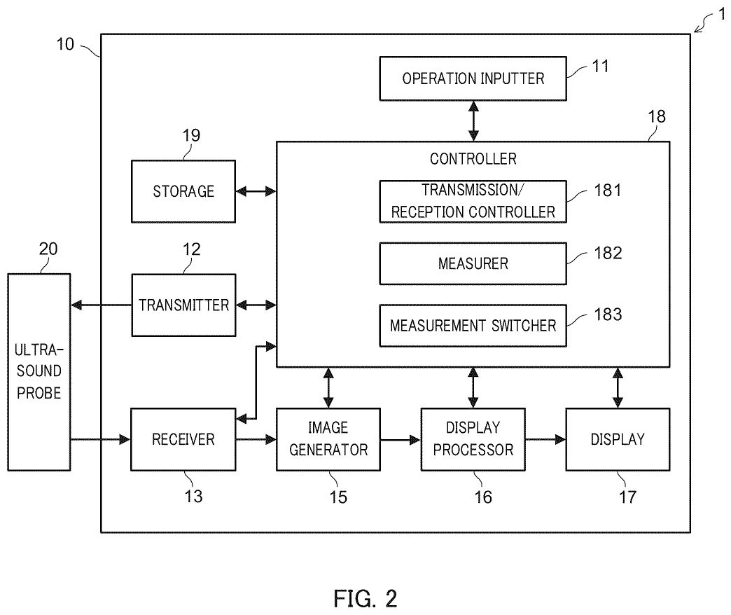

An ultrasound diagnostic apparatus according to the present embodiment will be described with reference to FIGS. 1 and 2. FIG. 1 is an external view of ultrasound diagnostic apparatus 1 as one example of an ultrasound diagnostic apparatus according to the present embodiment. FIG. 2 is a block diagram illustrating an example of a main part of ultrasound diagnostic apparatus 1.

Ultrasound diagnostic apparatus 1 is used for visualizing. as an ultrasound image, the shape, property. or dynamics of a biological tissue inside a subject such as a living body, and diagnosing the image.

As illustrated in FIG. 1, ultrasound diagnostic apparatus 1 includes apparatus main body 10, ultrasound probe 20, cable 30, and the like. In addition, apparatus main body 10 is provided with operation inputter 11, display 17, and the like which will be described later. In ultrasound diagnostic apparatus 1, ultrasound probe 20 is connected to apparatus main body 10 via cable 30. Ultrasound probe 20 may be connected to apparatus main body 10 via wireless communication.

### Apparatus Main Body

As illustrated in FIG. 2, apparatus main body 10 includes operation inputter 11, transmitter 12, receiver 13, image generator 15, display processor 16, display 17, controller 18, storage 19, and the like.

Operation inputter 11 (the inputter in the present invention) is a user interface for a user (e.g., a healthcare professional such as a doctor or a laboratory technician) to perform an input operation, converts the input operation performed by the user into an operation signal, and inputs the operation signal to controller 18. Operation inputter 11 includes, for example, an operation panel having a plurality of operation buttons, a keyboard, and a mouse. When a touch panel display is used as display 17, the touch panel portion functions as a part of operation inputter 11.

With operation inputter 11, the user performs, for example, input operations such as an operation at the time of measurement of ultrasound diagnostic apparatus 1, an operation at the time of diagnosis, and an operation for information to be displayed on display 17.

Transmitter 12 is a transmission circuit that generates a voltage pulse that is a drive signal, and outputs the voltage pulse to each acoustic element of ultrasound probe 20. Transmitter 12 sets the voltage amplitude, the pulse width, and the timing of the voltage pulse for each channel (each acoustic element).

For example, transmitter 12 changes a delay time of a voltage pulse to be supplied to each acoustic element so that an ultrasound output from each acoustic element is focused on a focal point set in the subject (delay processing).

In addition, transmitter 12 scans the inside of the subject with ultrasound by sequentially driving the acoustic elements by channel switching.

Receiver 13 is a reception circuit that receives and processes a reception signal generated by each acoustic element by reception of an ultrasound echo due to reflection of an ultrasound.

Receiver 13 amplifies and A/D converts the reception signal of each channel, and provides a delay time to the reception signal after the A/D conversion. At this time, receiver 13 gives a delay time so that the phases of the reception signals generated by the ultrasound echoes from the focal point set in the subject are aligned (delay processing). As a result, receiver 13 combines the reception signals of the plurality of channels into one (hereinafter, referred to as a “phasing addition signal”). Then, receiver 13 performs filter processing on the phasing addition signal and outputs the signal to image generator 15.

Image generator 15 performs generation processing of image data (hereinafter, ultrasound image data) of an ultrasound image to be two dimensional frame data, based on the phasing addition signals output from receiver 13. Image generator 15 generates ultrasound image data such as a B-mode image, a color Doppler image, or a three dimensional ultrasound image, for example, according to the scanning mode.

Image generator 15 generates the ultrasound image data after performing, for example, logarithmic compression processing, detection processing, FFT analysis processing, and the like. Since processing for generating ultrasound image data in image generator 15 may be similar processing to known processing, description thereof is omitted herein. The generated ultrasound image data may be stored in, for example, storage 19.

Display processor 16 converts the ultrasound image data generated by image generator 15 into a display signal corresponding to display 17, and outputs the display signal as display data.

Display 17 is configured by, for example, a liquid crystal display, an organic EL display, a CRT display. or a touch panel display. Display 17 displays the display data input from display processor 16 as an ultrasound image.

Controller 18 is a so-called computer, and although not illustrated, includes a CPU (Central Processor) as an arithmetic/control device, a ROM (Read Only Memory) and a RAM (Random Access Memory) as a main storage device, and the like. Controller 18 includes one or more hardware processors.

Programs and setting data are stored in the non-transitory computer-readable recording media, and the ROM stores the programs and setting data from the recording media. The CPU reads a program according to processing contents from the ROM, develops the program in the RAM, and executes the developed program to centrally control an operation of each functional block of ultrasound diagnostic apparatus 1. That is, controller 18 controls the entire ultrasound diagnostic apparatus 1 by controlling operation inputter 11, transmitter 12, receiver 13, image generator 15, display processor 16, and display 17 in accordance with their functions.

For example, controller 18 includes transmission/reception controller 181. Transmission/reception controller 181 controls transmitter 12 and receiver 13 to cause the acoustic element to be driven to execute transmission/reception processing of an ultrasound as described above.

Controller 18 includes measurer 182 and measurement switcher 183. Measurer 182, which will be described in detail later, includes a plurality of measurers that perform respective measurement processes. Each of the measurers automatically or manually performs measurement on the ultrasound image data, for example, measurement of the blood flow rate, the blood vessel diameter, and the like. Although details will be described later, measurement switcher 183 also switches between a plurality of measurers, for example, a first measurer and a second measurer to be described later.

Then, as will be described later, ultrasound diagnostic apparatus 1 switches the measurer under the control of measurer 182 and measurement switcher 183, and performs measurement on the ultrasound image data.

Storage 19 is, for example, a storage device such as a hard disk drive (HDD), a solid state drive (SSD), or a flash memory, and stores ultrasound image data generated by image generator 15 and information (a region of interest, a measurement position of a feature, and the like) detected and measured by measurer 182.

Transmitter 12, receiver 13, image generator 15, and display processor 16 described above may be configured by dedicated or general-purpose hardware (electronic circuit) corresponding to each process. and realize each function in cooperation with controller 18.

For example, transmitter 12, receiver 13, image generator 15, and display processor 16 are formed with hardware such as an application specific integrated circuit (ASIC), a digital signal processor (DSP), or a programmable logic device (PLD). The PLD includes a field programmable gate array (FPGA) or the like. In a case where transmitter 12 and receiver 13 are formed with a DSP, a PLD, or the like, they have a processing program therefor.

Furthermore, transmitter 12, receiver 13, image generator 15, and display processor 16 may be configured so that a CPU or a graphics processor (GPU) executes processing in each of the units. In this case, the CPU or the GPU has a processing program for performing processing of each unit.

### Ultrasound Probe

Ultrasound probe 20 is, for example, a convex probe, a linear probe, a sector probe, a three dimensional probe, or the like. Although illustration is omitted, ultrasound probe 20 includes a plurality of acoustic elements and the like. Each acoustic element is, for example, a piezoelectric element capable of converting an electrical signal into mechanical vibration and converting mechanical vibration into an electrical signal and capable of transmitting and receiving ultrasound.

Ultrasound probe 20 converts the voltage pulse output from transmitter 12 into an ultrasound, transmits the ultrasound into the subject, receives an ultrasound echo reflected inside the subject, and outputs the received reception signal to receiver 13. At this time, as described above, ultrasound probe 20 scans the inside of the subject with ultrasound by sequentially driving the acoustic elements by channel switching. Then, based on the received reception signals, image generator 15 generates ultrasound image data to be two dimensional frame data.

### Automatic Measurement and Manual Measurement

Ultrasound diagnostic apparatus 1 has the configuration described above. When a user brings ultrasound probe 20 into contact with a surface (skin) of the subject, ultrasound diagnostic apparatus 1 acquires ultrasound image data inside the subject and displays display data based on the acquired ultrasound image data as an ultrasound image on display 17. Since the ultrasound image data is generated as the two dimensional frame data by image generator 15, the ultrasound image can be displayed in real time by displaying the display data corresponding to the frame data in real time in time series. In addition, ultrasound diagnostic apparatus 1 can measure, for example, a blood flow rate, a blood vessel diameter, or the like by performing measurement on ultrasound image data in response to a user's operation.

As described above, in the field of ultrasound diagnostic apparatuses, there has been developed a real-time automatic measurement technique for automatically measuring, for example, a blood vessel diameter in real time without freezing an ultrasound image displayed in real time.

However, in the case of the conventional real-time automatic measurement, there has been a problem that automatic measurement processing (region detection, boundary detection, and the like) is continued indefinitely if the automatic measurement does not succeed due to an unclear ultrasound image or the like.

Therefore, in the present embodiment, in controller 18, measurer 182 includes a plurality of measurers, specifically, a first measurer and a second measurer. Measurement switcher 183 is configured to perform switching between the first measurer and the second measurer. In particular, measurement switcher 183 enables switching to the second measurer at a timing when the real-time automatic measurement performed by the first measurer is not completed, and enables the second measurement performed by the second measurer.

Here, first, the first measurer and the second measurer will be described below.

The first measurer performs real-time automatic measurement on the ultrasound image data.

The real-time automatic measurement is automatic measurement of an object in real time. The real-time automatic measurement includes measurement of a target by performing arithmetic processing together with generation of ultrasound image data. The real-time automatic measurement includes measuring the target by immediately performing arithmetic processing of the ultrasound image data every time the ultrasound image data is generated. In addition, the real-time automatic measurement includes, together with generation of the ultrasound image to be displayed, analysis of the generated ultrasound image to measure the target. The real-time automatic measurement is preferably performed without freezing the ultrasound image data.

The automatic measurement includes full-automatic measurement and semi-automatic measurement. The full-automatic measurement means to detect a measurement target and a measurement position from an ultrasound image and perform measurement processing without user's operation on the ultrasound image. The semi-automatic measurement is to detect a measurement position and perform measurement processing without a user operation after a measurement target or a measurement timing (e.g., diastole and systole in a case where a heart is a measurement target) is specified by a user for an ultrasound image.

Therefore, the real-time automatic measurement includes real-time full-automatic measurement (automatic measurement A1) and real-time semi-automatic measurement (automatic measurement A2).

That is, automatic measurement A1 generates an ultrasound image to be displayed, detects a measurement position, and performs measurement processing on the ultrasound image generated in real time without a user's operation.

Automatic measurement A2 detects a measurement position and performs measurement processing on an ultrasound image generated and displayed in real time without a user's operation after the user designates a measurement target and measurement timing.

The real-time automatic measurement detects a region of interest to be a measurement target or a feature for the region of interest to be the measurement target in ultrasound image data to be generated or an ultrasound image to be displayed. The detection of the feature includes detection of a measurement position.

Detection of a region of interest that is a measurement target by real-time automatic measurement is, for example, detection of the left ventricle of the heart, a blood vessel, a fetus, or the like. In addition, the feature detection for the region of interest that is a measurement target in the real-time automatic measurement is, for example, detection of a left ventricle ejection fraction of the heart, a measured value of a left ventricle stroke volume of the heart, a velocity-time integral value of a left ventricle outflow tract of the heart, a measured value of a blood vessel diameter, a measured value of a fetal head-buttocks length, and the like. The measurement value of the blood vessel diameter includes measurement values of the inferior vena cava, the carotid artery, the brachial artery, and the like.

The second measurer performs second measurement different from the real-time automatic measurement. The second measurement includes a case of manually measuring by freezing ultrasound image date (manual measurement B1), a case of manually measuring in real time (manual measurement B2), and a case of automatically measuring by freezing ultrasound image date (measurements B3 and B4).

The manual measurement includes, for example, designating a measurement target image, a region of interest serving as a measurement target, and a measurement position with respect to a displayed ultrasound image by a user using operation inputter 11 and performing measurement based on the measurement target image, the region of interest, and the measurement position designated by the user.

That is, manual measurement B1 freezes the ultrasound image, and the user designates the measurement target image, the region of interest to be the measurement target, and the measurement position with respect to the currently displayed ultrasound image by using operation inputter 11. After the designation, manual measurement B1 performs measurement based on the measurement target image, the region of interest, and the measurement position designated by the user.

In manual measurement B2, the user designates a measurement target image, a region of interest to be a measurement target, and a measurement position with respect to an ultrasound image generated and displayed in real time using operation inputter 11. After the designation, manual measurement B2 performs measurement based on the measurement target image, the region of interest, and the measurement position designated by the user.

Measurement B3 is a case where ultrasound image data is frozen and full-automatic measurement is performed. In measurement B3, ultrasound image data is frozen, and a measurement position is detected and measurement processing is performed on a currently displayed ultrasound image without a user operation.

Measurement B4 is a case where ultrasound image data is frozen and semi-automatic measurement is performed. In measurement B4, the ultrasound image is frozen, and the user specifies the measurement target and the measurement timing for the currently displayed ultrasound image using operation inputter 11. After the designation, measurement B4 detects a measurement position and performs measurement processing without a user's operation.

As described above, measurer 182 includes the first measurer that performs real-time automatic measurement and the second measurer that performs second measurement. Measurement switcher 183 enables the second measurement performed by the second measurer at a timing when the real-time automatic measurement performed by the first measurer is not completed. That is, during the execution of the real-time automatic measurement by the first measurer, measurement switcher 183 can shift, that is, switch to the second measurement performed by the second measurer at a timing when the measurement is not completed.

### Measurement Switching

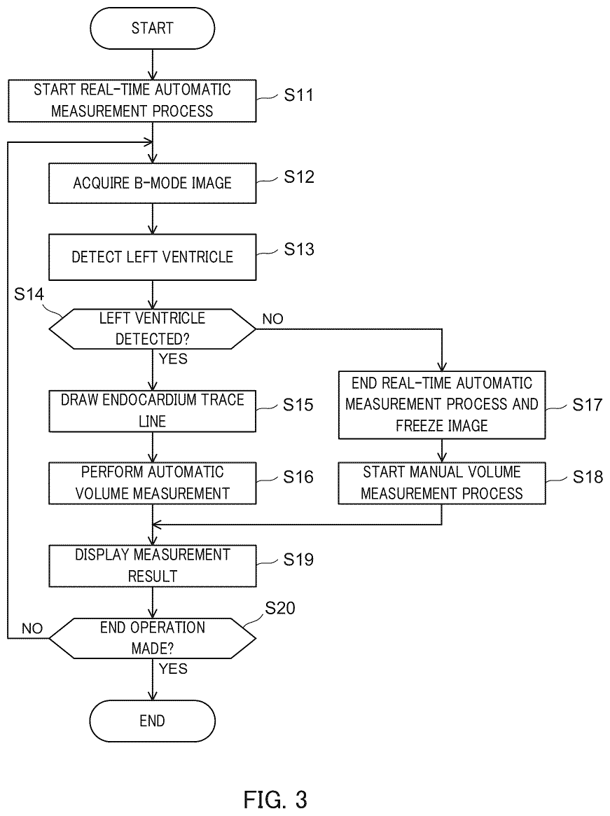

An example of control (measurement switching) of ultrasound diagnostic apparatus 1 including measurer 182 and measurement switcher 183 will be described with reference to FIG. 3. FIG. 3 is a flowchart illustrating an example of a control method of ultrasound diagnostic apparatus 1.

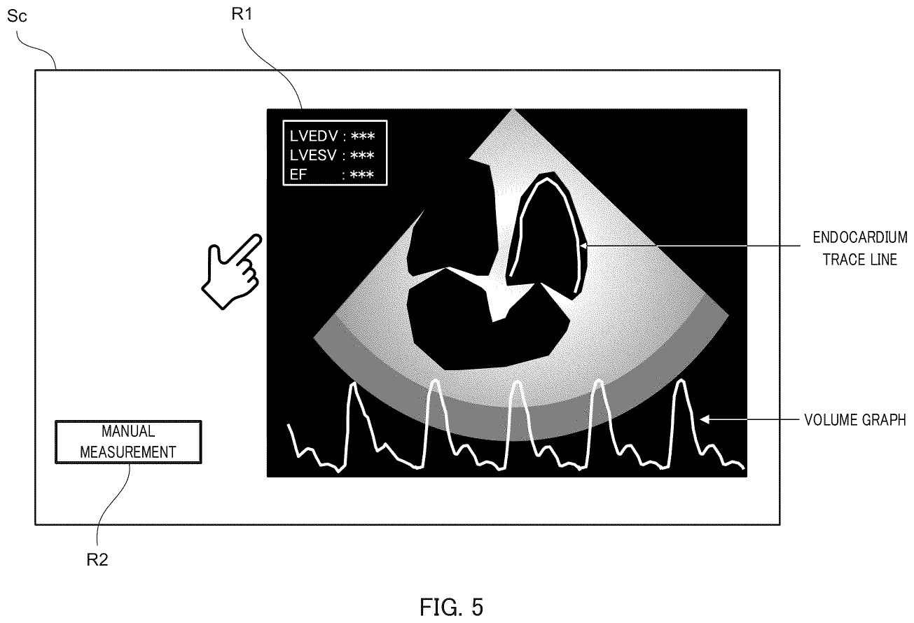

Note that here, as an example, the description will be given taking as an example the case where ultrasound diagnostic apparatus 1 acquires a B-mode image as ultrasound image data, detects the left ventricle as a region of interest, detects the endocardium as a feature, and draws endocardium trace lines (refer to FIG. 5 described later).

### Step S11

Controller 18 starts the real-time automatic measurement process, for example, by the operation of the user from operation inputter 11. At this time, controller 18 controls measurer 182 to activate the first measurer that performs real-time automatic measurement processing (automatic measurements A1 and A2).

### Step S12

Controller 18 controls transmitter 12 and receiver 13 via transmission/reception controller 181 to transmit and receive ultrasound, and controls image generator 15 to acquire image data of a B-mode image.

### Step S13

Controller 18 controls measurer 182 to detect the left ventricle (region of interest) without user operation on the B-mode image generated in real time. At this time, controller 18 also controls display processor 16 to create display data of the B-mode image in real time and output it to display 17, so that the B-mode image is displayed on display 17.

### Step S14

Controller 18 checks whether the left ventricle can be detected. and proceeds to step S15 if the left ventricle can be detected (YES), or proceeds to step S17 if the left ventricle cannot be detected (NO). For example, when an error or the like occurs in measurer 182 and the left ventricle cannot be detected, controller 18 proceeds to step S17.

Note that here, the left ventricle is set as a detection target, but the endocardium may be set as a detection target, and in a case where the endocardium can be detected (YES), the process may proceed to step S15, and in a case where the endocardium cannot be detected (NO), the process may proceed to step S17.

### Step S15

Controller 18 controls measurer 182 to detect the endocardium (measurement positions of the measurement target) and draw endocardium trace lines based on the detection result of the endocardium. At this time, controller 18 also controls display processor 16 to create display data of endocardium trace lines and output the display data to display 17, so that display 17 displays the endocardium trace lines together with the B-mode image.

### Step S16

Controller 18 controls measurer 182 to perform automatic volume measurement based on the endocardium trace lines and calculate the volume of the left ventricle.

### Step S17

If the left ventricle cannot be detected in step S14 (NO), controller 18 controls measurer 182 to end the real-time automatic measurement process and freeze the ultrasound image currently displayed on display 17. That is, before the detection of the left ventricle (or endocardium) is completed, controller 18 controls measurer 182 to end the real-time automatic measurement process and freeze the ultrasound image currently displayed on display 17.

At this time, the ultrasound image data corresponding to the currently displayed B-mode image is also frozen. At this time, if there is information that can be acquired by the real-time automatic measurement, for example, if the endocardium cannot be detected but the left ventricle can be detected, the position information and the like may be stored in storage 19 and handed over to the second measurer. In this case, for example, when part of the result of the real-time automatic measurement is correct but part of the result is incorrect, part of the result may be stored in storage 19 and part of the result may be corrected by the user using operation inputter 11.

In this way, the second measurer may take over the information acquired by the real-time automatic measurement to perform the second measurement. In this case, for example, the user may specify information on some of the measurement target image, the region of interest, and the measurement position in manual measurement B1 described below, which can reduce the burden on the user.

### Step S18

Controller 18 controls measurement switcher 183 to switch from the first measurer to the second measurer, controls measurer 182 to activate the second measurer, and starts the second measurement for performing the manual volume measurement process.

Here, the above-described manual measurement B1 is used as the manual volumetric measurement processing. That is, the ultrasound image data is frozen, and the user specifies, on the currently displayed B-mode image, the measurement target image and the measurement positions of the left ventricle and the endocardium that are the region of interest that is a measurement target, using operation inputter 11. After the designation, manual measurement B1 calculates the left ventricle volume based on the measurement target image, the region of interest, and the measurement position designated by the user.

Note that here, manual measurement B1 is used as the second measurement, but instead of this, the above-described manual measurement B2, measurement B3, and B4 may be used.

### Step S19

Controller 18 controls display processor 16 to generate display data on the measured volume of the left ventricle and output the display data to display 17, so that display 17 displays the volume of the left ventricle together with the B-mode image and the endocardium trace line.

### Step S20

Controller 18 confirms whether or not there is an end operation from operation inputter 11, and in a case where there is an end operation (YES), ends the series of processes, and in a case where there is no end operation (NO), returns to step S12. That is, steps S12 to S20 are repeated until an end operation is performed.

Under the above-described control, ultrasound diagnostic apparatus 1 basically performs real-time automatic measurement, but when a region of interest or a feature cannot be detected, that is, when automatic measurement does not succeed, the automatic measurement processing can be stopped before the completion of automatic measurement. Then, when the automatic measurement processing is stopped, the processing automatically shifts to manual measurement. As a result, it is possible to prevent the problem of continuing the automatic measurement processing indefinitely when the automatic measurement is not successful, and necessary measurement can be continuously performed by manual measurement.

In addition, in the above-described control, as shown in the flowchart of FIG. 3, since the process returns to the control loop of the automatic measurement even when the process shifts to the manual measurement once, the real-time automatic measurement is basically performed, when automatic measurement cannot be performed, manual measurement is performed. Further, since the measurement result in the automatic measurement and the measurement result in the manual measurement are stored in storage 19 so as to be taken over by each other, it is possible to reduce the burden on the user.

In the above-described control, when the left ventricle or the endocardium cannot be detected, the process shifts to the manual measurement before the detection is completed. However, when measurer 182 cannot calculate the automatic volume measurement due to an error or the like, the process may shift to the manual measurement before the calculation is completed.

### Modification 1

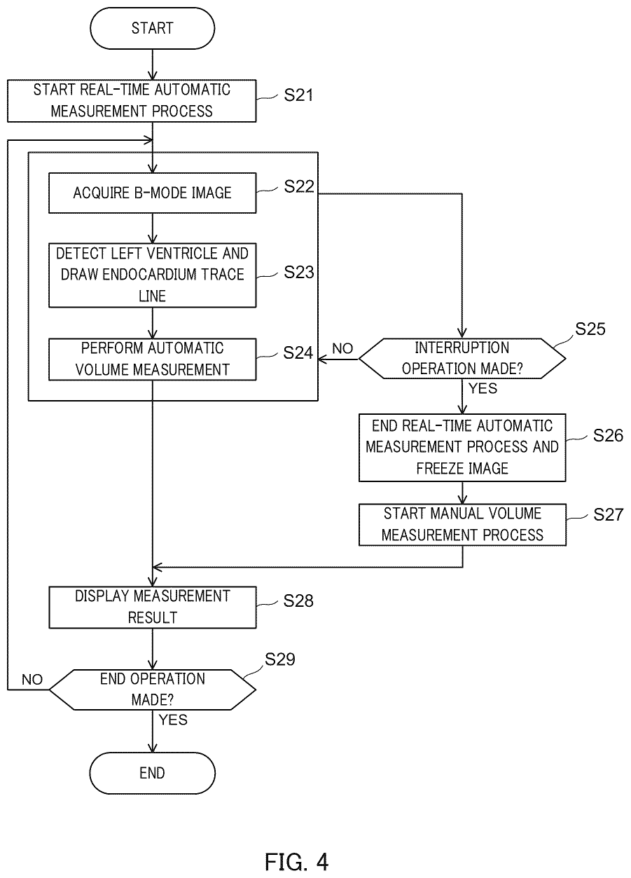

Another example of the control (measurement switching) of ultrasound diagnostic apparatus 1 will be described with reference to FIG. 4. FIG. 4 is a flowchart for explaining another example of the control method of ultrasound diagnostic apparatus 1.

In this modification, ultrasound diagnostic apparatus 1 described with reference to FIGS. 1 and 2 is used as the ultrasound diagnostic apparatus. Therefore, redundant description of the configuration of ultrasound diagnostic apparatus 1 will be omitted.

In FIG. 3, controller 18 determines whether detection of a region of interest or a feature in automatic measurement is successful, and stops automatic measurement processing if the detection of the region of interest or the feature is not successful. In contrast, in the present modification, controller 18 determines whether or not there is an interruption operation from operation inputter 11 during the automatic measurement, and when there is an interruption operation, the automatic measurement process is stopped. Such control of the present modification example will be described below.

Note that also here, as an example, a case will be described in which ultrasound diagnostic apparatus 1 acquires a B-mode image as ultrasound image data, detects the left ventricle as a region of interest, detects the endocardium as a feature, and draws endocardium trace lines (see FIG. 5 described later).

### Step S21

Since step S21 is the same processing as step S11 described above, description thereof is omitted.

### Step S22

Step S22 is also the same processing as step S12 described above, and therefore, description thereof is omitted.

### Step S23

Controller 18 controls measurer 182 to detect the left ventricle and the endocardium (the measurement position of the measurement target) from the B-mode image generated in real time without the user's operation, and draws the endocardium trace line based on the detection result of the endocardium. At this time, controller 18 also controls display processor 16 to create display data of the B-mode image in real time and also create display data of the endocardium trace line and output the display data to display 17, thereby causing display 17 to display the endocardium trace line together with the B-mode image.

### Step S24

Since step S24 is the same processing as step S16 described above, the description thereof will be omitted.

In the present modification example, while controller 18 executes the above-described steps S22 to S24, it can receive an input from operation inputter 11 constantly or at a predetermined timing. Therefore, when there is an input from operation inputter 11 in steps S22 to S24, the process proceeds to step S25.

### Step S25

When there is an input from operation inputter 11, controller 18 checks whether or not there is an interruption operation of the real-time automatic measurement processing. Controller 18 proceeds to step S26 when there is an interruption operation of the real-time automatic measurement processing (YES), and continues the processing in steps S22 to S24 as it is when there is no interruption operation (NO).

Here, the interruption operation will be described with reference to FIG. 5. FIG. 5 is a diagram illustrating an interruption operation in the flowchart illustrated in FIG. 4.

In FIG. 5, screen Sc is a screen of display 17 displaying a B-mode image of the heart using a sector probe as ultrasound probe 20. In display region R1 of the B-mode image in screen Sc, an endocardium trace line is displayed on the left ventricle, and changes in the left ventricle volume are displayed as a graph. The volume of the left ventricle is calculated using, for example, the Simpson method.

When starting the real-time automatic measurement processing, controller 18 controls display processor 16 to display the operation region R2 of “manual measurement” on the screen S constantly or at a predetermined timing. Then, when the user clicks (touches in the case of the touch panel) the operation area R2, controller 18 determines that the interruption operation has been performed.

In addition, when controller 18 starts the real-time automatic measurement process, if the user clicks (touches in the case of a touch panel) the display region R1 of the B-mode image, it may determine that the interruption operation is performed.

### Steps S26 to S29

Steps S26 to S29 are the same processing as the above-described steps S17 to S20, and thus description thereof will be omitted.

According to the above-described control, ultrasound diagnostic apparatus 1 basically performs real-time automatic measurement in the present modification, but can stop the automatic measurement processing when the user performs an interruption operation. Then, when the automatic measurement processing is stopped, the processing automatically shifts to manual measurement. In this way, it is possible to prevent the problem that the automatic measurement processing is continued indefinitely by the interruption operation of the user, and necessary measurement can be continuously performed by manual measurement.

### Modification 2

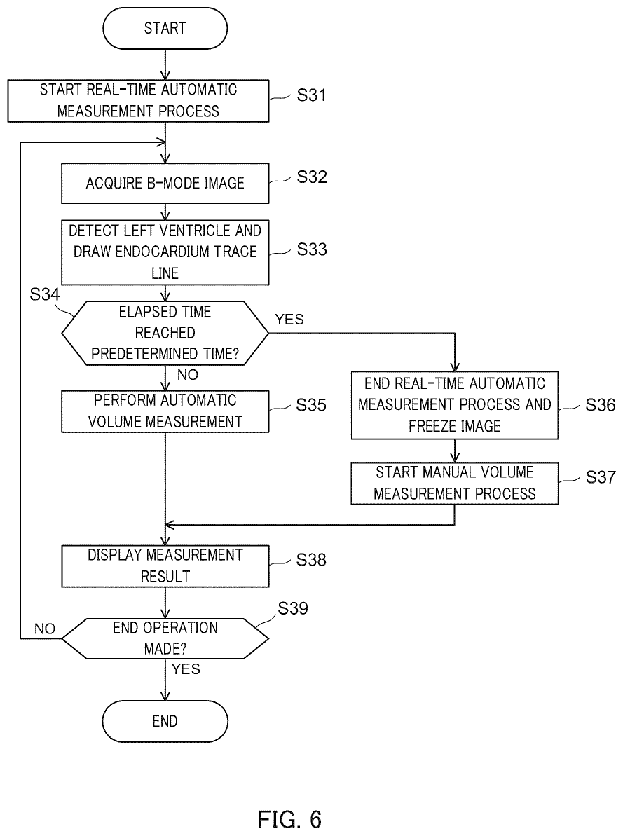

Another example of the control (measurement switching) of ultrasound diagnostic apparatus 1 will be described with reference to FIG. 6. FIG. 6 is a flowchart illustrating another example of the control method of ultrasound diagnostic apparatus 1.

Also in the present modification example, ultrasound diagnostic apparatus 1 described with reference to FIG. 1 and FIG. 2 is used as the ultrasound diagnostic apparatus. Therefore, redundant description of the configuration of ultrasound diagnostic apparatus 1 will be omitted.

In FIG. 3, controller 18 determines whether detection of a region of interest or a feature in automatic measurement is successful, and stops automatic measurement processing if the detection of the region of interest or the feature is not successful. Further, in FIG. 4, controller 18 determines whether or not there is an interruption operation from operation inputter 11 during automatic measurement, and when there is an interruption operation, the automatic measurement processing is stopped. In contrast, in the present modification example, controller 18 monitors the time taken for automatic measurement (e.g., the time taken for detection of a region of interest or a feature) and stops automatic measurement processing when the time has exceeded a predetermined time. Such control of the present modification example will be described below.

Note that also here, as an example, the description will be given taking as an example the case where ultrasound diagnostic apparatus 1 acquires a B-mode image as ultrasound image data. detects the left ventricle as a region of interest, detects the endocardium as a feature, and draws endocardium trace lines (refer to FIG. 5).

### Steps S31 to S33

Steps S31 to S33 are the same processing as the above-described steps S21 to S23, and thus description thereof will be omitted.

### Step S34

Controller 18 measures, in steps S32 to S33, an elapsed time from the acquisition of the B-mode image to the detection of the left ventricle and the endocardium and the drawing of the endocardium trace lines. Then, controller 18 checks whether or not the elapsed time has reached the predetermined time. When the elapsed time has reached the predetermined time (YES), the process proceeds to step S36, and when the elapsed time has not reached the predetermined time (NO), the process proceeds to step S35.

### Step S35

Since step S35 is the same processing as step S24 described above, description thereof is omitted.

### Steps S36 to S39

Steps S36 to S39 are the same processing as the above-described steps S26 to S29, and thus description thereof will be omitted.

Through the above-described control, ultrasound diagnostic apparatus 1 basically performs real-time automatic measurement, but can stop automatic measurement processing when it takes time to detect a region of interest or a feature, that is, when automatic measurement does not succeed. Then, when the automatic measurement processing is stopped, the processing automatically shifts to manual measurement. As a result, it is possible to prevent the problem of continuing the automatic measurement processing indefinitely when the automatic measurement is not successful, and necessary measurement can be continuously performed by manual measurement.

In the above-described control, in a case where it takes time to detect the left ventricle or the endocardium, the process proceeds to the manual measurement before the detection is completed. However, in a case where it takes time for measurer 182 to calculate the automatic volume measurement due to an error or the like, the process may proceed to the manual measurement before the calculation is completed.

The above-described embodiments are merely examples for implementing the present invention, and the technical scope of the present invention should not be interpreted in a limited manner by these embodiments. That is, the present invention can be implemented in various forms without departing from the spirit or main features thereof.

Although embodiments of the present invention have been described and illustrated in detail, the disclosed embodiments are made for purpose of illustration and example only and not limitation. The scope of the present invention should be interpreted by terms of the appended claims.

What is claimed is:

1. An ultrasound diagnostic apparatus comprising:
an ultrasound probe configured to transmit and receive ultrasound to and from a subject; and
one or more hardware processors;
wherein the hardware processor
generates image data based on a reception signal received by the ultrasound probe,
performs first measurement of performing real-time automatic measurement on the image data, and
enables second measurement different from the real-time automatic measurement at a timing when the real-time automatic measurement performed in the first measurement has not been completed.

2. The ultrasound diagnostic apparatus according to claim 1, wherein the real-time automatic measurement includes detection of a region of interest that is a measurement target.

3. The ultrasound diagnostic apparatus according to claim 1, wherein the real-time automatic measurement includes feature detection for a region of interest that is a measurement target.

4. The ultrasound diagnostic apparatus according to claim 1, wherein the second measurement is manual measurement with the image date frozen.

5. The ultrasound diagnostic apparatus according to claim 1, wherein the hardware processor enables the second measurement before detection of a region of interest that is a measurement target is completed in the first measurement or before feature detection for the region of interest is completed in the first measurement.

6. The ultrasound diagnostic apparatus according to claim 1, wherein the hardware processor enables the second measurement when it is determined that a region of interest that is a measurement target cannot be detected in the first measurement or when it is determined that feature detection for the region of interest cannot be detected in the first measurement.

7. The ultrasound diagnostic apparatus according to claim 1, wherein the hardware processor enables the second measurement before a result of the real-time automatic measurement is calculated in the first measurement.

8. The ultrasound diagnostic apparatus according to claim 1, further comprising an inputter configured to receive a predetermined input operation,
wherein the hardware processor enables the second measurement at a timing after the predetermined input operation is performed.

9. The ultrasound diagnostic apparatus according to claim 1, wherein the hardware processor enables the second measurement at a timing after a predetermined time has elapsed.

10. The ultrasound diagnostic apparatus according to claim 1, wherein at a timing when the real-time automatic measurement performed in the first measurement is not completed, the hardware processor ends the real-time automatic measurement and enables switching to second measurement different from the real-time automatic measurement.

11. The ultrasound diagnostic apparatus according to claim 2, further comprising a storage configured to store the region of interest,
wherein the hardware processor performs the second measurement based on the region of interest stored in the storage.

12. The ultrasound diagnostic apparatus according to claim 11, wherein the storage stores a feature for the region of interest, and
the hardware processor performs the second measurement based on the region of interest and the feature stored in the storage.

13. The ultrasound diagnostic apparatus according to claim 12, further comprising an inputter with which a user designates at least one of a region of interest and a measurement position as a measurement target for the image data,
wherein the hardware processor performs the second measurement based on the region of interest and the feature stored in the storage, and at least one of the region of interest and the measurement position designated by the user.

14. A non-transitory computer-readable recording medium storing an ultrasound diagnostic program for causing a computer to execute:
transmission/reception processing of transmitting and receiving ultrasound to and from a subject with an ultrasound probe;
generation processing of generating image data based on a reception signal received by the ultrasound probe;
first measurement processing of performing first measurement of performing real-time automatic measurement on the image data; and
second measurement processing of enabling second measurement different from the real-time automatic measurement at a timing when the real-time automatic measurement performed in the first measurement is not completed.

15. The recording medium according to claim 14, wherein the real-time automatic measurement includes detection of a region of interest that is a measurement target.

16. The recording medium according to claim 14, wherein the real-time automatic measurement includes feature detection for a region of interest that is a measurement target.

17. The recording medium according to claim 14, wherein the second measurement is manual measurement with the image date frozen.

18. The recording medium according to claim 14, wherein the second measurement processing enables the second measurement before the first measurement processing completes detection of a region of interest that is a measurement target or before the first measurement processing completes feature detection for the region of interest.

19. The recording medium according to claim 14, wherein the second measurement processing enables the second measurement when it is determined that the first measurement processing cannot detect a region of interest that is a measurement target or when it is determined that the first measurement processing cannot perform feature detection on the region of interest.

20. The recording medium according to claim 14, wherein the second measurement processing enables the second measurement before a result of the real-time automatic measurement by the first measurement processing is calculated.

21. The recording medium according to claim 14, wherein the second measurement processing enables the second measurement at a timing after a predetermined input operation is performed.

22. The recording medium according to claim 14, wherein the second measurement processing enables the second measurement at a timing after a predetermined time has elapsed.

23. The recording medium according to claim 14, wherein at a timing when the real-time automatic measurement performed in the first measurement processing is not completed. the second measurement processing ends the real-time automatic measurement and enables switching to second measurement different from the real-time automatic measurement.

24. The recording medium according to claim 15, wherein the second measurement processing performs the second measurement based on a region of interest stored in a storage.

25. The recording medium according to claim 24, wherein the second measurement processing performs the second measurement based on the region of interest and a feature stored in the storage.

26. The recording medium according to claim 25, wherein the second measurement processing performs the second measurement based on the region of interest and the feature stored in the storage, and at least one of a region of interest and a measurement position designated as a measurement target for the image data by a user.

27. A control method of an ultrasound diagnostic apparatus configured to transmit and receive ultrasound to and from a subject with an ultrasound probe and generate image data based on a reception signal received by the ultrasound probe, the control method comprising:
performing first measurement of performing real-time automatic measurement on the image data; and
enabling second measurement different from the real-time automatic measurement at a timing when the real-time automatic measurement performed in the first measurement is not completed.
